(19) United States
(12) Patent Application Publication
(10) Pub. No.: US 2011/0258760 A1
(43) Pub. Date: Oct. 27, 2011

(54) PROTECTIVE MASK FOR THE PRACTICE OF OUTDOOR SPORTS

(75) Inventors: Gilles RENAUD-GOUD, Gruffy (FR); Quentin VERHAEGHE, Annecy (FR)

(73) Assignee: SALOMON S.A.S., Metz-Tessy (FR)

(21) Appl. No.: 13/091,642

(22) Filed: Apr. 21, 2011

(30) Foreign Application Priority Data
Apr. 22, 2010 (FR) 10.01728

(51) Int. Cl. A61F 9/02

(52) U.S. Cl. 2/431

(57) ABSTRACT

The invention relates to an eye protection mask for the practice of outdoor sports, the mask including an eye protection lens having two vision zones, adapted to be positioned opposite the user's eyes when worn, and a median portion connecting the vision zones, and a peripheral frame demarcating an opening closed by the lens, the frame having a groove housing the edge of the lens, the frame being made of a rigid material and the groove having a depth of less than 2.5 mm along the vertical direction of the vision zones of the lens.

### CROSS-REFERENCE TO RELATED APPLICATION

This application is based upon the French priority Patent Application No. 10.01728, filed Apr. 22, 2010, the disclosure of which is hereby incorporated by reference thereto, and the priority of which is hereby claimed under 35 U.S.C. §119.

### BACKGROUND OF THE INVENTION

1. Field of the Invention

The invention relates to protective masks, in particular protective masks for the practice of outdoor sports such as alpine skiing, mountain biking, and motocross. In more particular embodiments, the invention relates to protective eyewear, or goggles.

2. Background Information

Protective masks, such as protective eyewear, or goggles, are typically used in the practice of certain outdoor sports to protect the user's eyes from wind, water, mud and/or solar radiation. The use of such masks is generally favored over sunglasses with fully rimmed frames because of the larger continuous field of vision they provide, especially in the median portion. Furthermore, the use of such masks is also favored because they permit the user to wear prescription eyeglasses while enjoying the protection of the mask.

A mask of the aforementioned type usually includes a frame supporting a transparent protective shield, or lens. The protective lens is generally made of a synthetic material and is generally comprised of one or more continuous and transparent walls arranged to be opposite the user's eyes when worn. An outer wall of the lens can for example have solar radiation filtering properties (e.g., to reduce the penetration of ultraviolet radiation in the mask). An inner wall of the lens can have properties for limiting the condensation of water vapor. The lens is arranged in a front opening of the frame. The frame forms a peripheral support for the lens and generally supports a peripheral foam material that comes in contact with the user's face in order to adapt to the shape, or morphology, of the face. The frame and the foam keep the lens at a distance from the user's face, such that an inner volume is provided between the lens and the face. This inner volume can receive prescription eyeglasses, if worn by the user. The protective mask generally includes a strap attached to both sides of the frame. The frame and the strap thus form a loop, the strap passing behind the user's head to maintain the mask on the user's face.

A mask of this type generally has openings in the frame or in the lens to allow communication between the inner volume and the external environment. Such openings make it possible to evacuate water vapor from the inner volume, and thus to reduce condensation on the lens, especially when the user is perspiring. The frame is generally comprised of a structure made of flexible polymer materials, in order to dampen possible impacts against the mask, to facilitate the mounting of the lens, and to improve adaptation of the mask to the morphology of the user's face. The frame has a peripheral groove in which the edge of the lens is fitted or housed. Such a groove makes it possible to maintain the lens in position.

A mask of the aforementioned type has drawbacks. The frame must concurrently keep the lens in place, provide resistance to impacts, and adapt to the shape of the user's face. For these reasons, the frame is made of a flexible material. The frame has a relatively deep groove for housing the edge of the lens and a relatively deep width to ensure good retention of the lens. The height and width of the frame thus limit the viewing angle of the entire field of vision. Such a mask thus has a relatively reduced field of vision, in spite of a large lens.

U.S. Pat. No. 5,642,530 discloses such a mask, or goggles, in which the frame is made in a single piece. It includes a front portion in which a deep groove is provided, and a lateral wall whose base is provided with a strip of foam to absorb perspiration.

Similarly, U.S. Pat. No. 5,363,512 discloses a mask, or goggles, according to the prior art, the frame of which, made in a single piece, is soft and flexible.

The problem of reduced field of vision is accentuated when the mask is equipped with a double lens, or a double-walled lens, i.e., a front lens member and a rear lens member. An example of this is disclosed in EP 2 044 912 and family member U.S. Patent Application Publication No. 2009/0100577. FIG. 4 of this document shows that only one of the two lens members, i.e., the front lens member, is embedded within a deep groove of the frame; the second lens member, i.e., the rear lens member, is smaller than the front lens member, the two lens members being fixed together by a joint, i.e., a gasket. In such a case, in order to determine the vertical extension of the field of vision, the distance between the upper portion and the lower portion of the gasket must be measured. An estimate of the measurements on the mask, or goggles, disclosed in EP 2 044 912 shows that the vertical extension of the field of vision corresponds to 46% of the total height of the frame. In the hypothetical case in which the mask disclosed in EP 2 044 912 is to be equipped with a single-walled lens, the vertical extension of the field of vision is defined by the distance separating the two inner edges of the frame and corresponds to 64% of the outer vertical extent, or dimension, of the frame.

Therefore, the use of flexible materials for making the frame has a very negative effect on the field of vision/space requirement ratio of the mask. A study of various commercially available ski goggles has shown that the vertical extent, or dimension, of the field of vision remains less than 73% of the total height of the goggles for the currently available masks having a double-walled lens. If the frames of these goggles were to be equipped with a single walled lens, the ratio would still be less than 80%.

Furthermore, the aesthetics of the mask, or goggles, is strongly influenced by the shape or morphology of the user's face. In fact, the frame can undergo significant deformations in order to adapt to the morphology of the user's face, which can substantially change the appearance of the mask. Moreover, the vision of the user can also deteriorate because these deformations of the frame transferred to the lens, i.e., such deformations of the frame cause deformations of the lens. The shape of the lens, when it is worn, can be very different from its shape as designed to provide an optimum vision in the absence of stress on the mask. An impact against the mask can also generate an excessive deformation of the lens resulting in its rupture, or cracking.

Moreover, such a mask does not allow sufficient removal of moisture from the lens, or eliminate fogging of the lens, especially when the ambient humidity is high or the user is perspiring profusely. This is a particular problem when the user wears prescription eyeglasses that have not been treated against fogging. Increasing the section of the air vents proportionately reduces the field of vision of the user, which may generate unpleasant cooling on the face of the user without providing any efficiency in evacuating water vapor. Certain technical solutions have proposed to mount fans. Such fans however require a power supply, substantially weighing down the mask, substantially increasing the space requirement of the mask, and altering the field of vision that is provided.

### SUMMARY

The invention overcomes one or more of the aforementioned disadvantages. To this end, the invention relates to an eye protection mask, or goggles, for the practice of outdoor sports, including:
an eye protection lens oriented substantially perpendicular to a line of sight having two vision zones, the central portion of the vision zones being adapted to be positioned opposite the user's eyes, and a median portion connecting the vision zones;
a peripheral frame demarcating an opening closed by the lens, the frame having a groove housing the edge of the lens;
a deformable skirt attached to the frame extending behind the lens and demarcating an opening in alignment with the opening of the frame, such opening being adapted to be positioned on the user's face;
the frame being made of a rigid material; and
the distance separating the two inner edges of the frame in the area of the central portion of the vision zones being greater than 85% of the total height of the frame in the same area.

According to an alternative embodiment, the vertical extension of the field of vision in the area of the central portion of the vision zones is greater than 78% of the height of the frame in the same area.

According to an alternative embodiment, the groove has a vertical depth of less than 2.5 millimeters (mm) beyond the aforementioned vision zones of the lens.

According to an alternative embodiment, the frame has an edge, or rim, defining a front stop of the aforementioned groove, such edge having a thickness of less than 2.5 mm vertically of the aforementioned vision zones of the lens.

According to yet an alternative embodiment, the cross section of the frame has a height less than 6 mm vertically in the vision zones of the lens.

According to another alternative embodiment, the lens is snap-fitted into the groove of the frame.

According to yet another alternative embodiment, the groove extends over the entire periphery of the opening.

According to an alternative embodiment, the peripheral frame is made of a synthetic material having a modulus of elasticity greater than 1500 MPa.

According to another alternative embodiment, the frame has a hardness greater than 70 Shore D.

According to yet another alternative embodiment, the portion of the lens closing the opening of the frame is free of any through-opening, and the frame has a conduit, or passageway, opening out at the rear of the lens and arranged above the median portion of the lens.

According to another alternative embodiment, the mask includes a deflector arranged on the front surface of the frame, directing an axial air flow toward an outer inlet of the aforementioned passageway.

According to yet another alternative embodiment, the lens has two superimposed walls that are affixed by a peripheral joint, or connection, attached to the aforementioned groove.

According to an alternative embodiment, the mask includes a deformable skirt attached to the frame, extending behind the lens and demarcating an opening in alignment with the opening of the frame.

### BRIEF DESCRIPTION OF DRAWING

Other characteristics and advantages of the invention will become apparent from the description that follows, with reference to the annexed drawings illustrating, by way of non-limiting embodiments, how the invention can be embodied, and in which:

FIG. 1 is a perspective view of a mask according to an embodiment of the invention;

FIG. 2 is a top view of the mask of FIG. 1;

FIG. 3 is a front view of a portion of the mask of FIG. 1;

FIG. 4 is a side cross-sectional view of a method for attaching a lens to a frame;

FIG. 5 is a side cross-sectional view of the frame, in the area of a zone for attachment to the lens;

FIG. 6 is a side cross-sectional view of an improvement to the mask;

FIG. 7 illustrates air flow inward of the mask of FIG. 6;

FIG. 8 is a front view of the frame of an alternative mask;

FIG. 9 is a perspective view of the frame of FIG. 8, to which a deflector is fixed;

FIG. 10 is a perspective view of the deflector of FIG. 9;

FIG. 11 is a top cross-sectional view illustrating air flows generated by the deflector;

FIG. 12 is a perspective view of a frame according to an alternative mask;

FIG. 13 is a perspective view of the edge of a lens adapted to be attached to the frame of FIG. 12;

FIG. 14 is a side cross-sectional view of the fixing of the lens of FIG. 13 on the frame of FIG. 12;

FIG. 15 is a partial cross-sectional view along the plane C, shown in FIG. 1;

FIG. 16 is a vertical cross-sectional view of the mask of FIG. 1, in the area of the center of one of the two vision zones, such as along plane C;

FIG. 17 is a side view of the skirt of the mask of FIG. 1.

### DETAILED DESCRIPTION

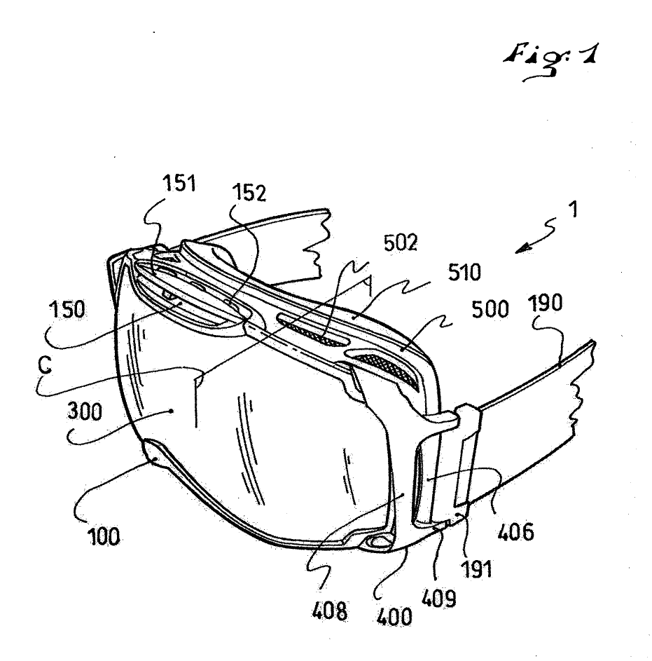

FIG. 1 is a perspective view of a mask 1 according to an embodiment of the invention. Although the term “mask” is used herein, the invention can be considered to relate to protective eyewear, or goggles, as well. FIG. 2 is a schematic top view of the mask 1. The mask 1 includes a peripheral frame 100. The frame 100 demarcates an opening 102, which is closed by an eye protection lens 300. The lens 300 is attached to the frame 100. A strap 190 is attached to the lateral ends of the mask 1. The strap 190 and frame 100 form a loop for surrounding the head of the user to ensure that it stays in place.

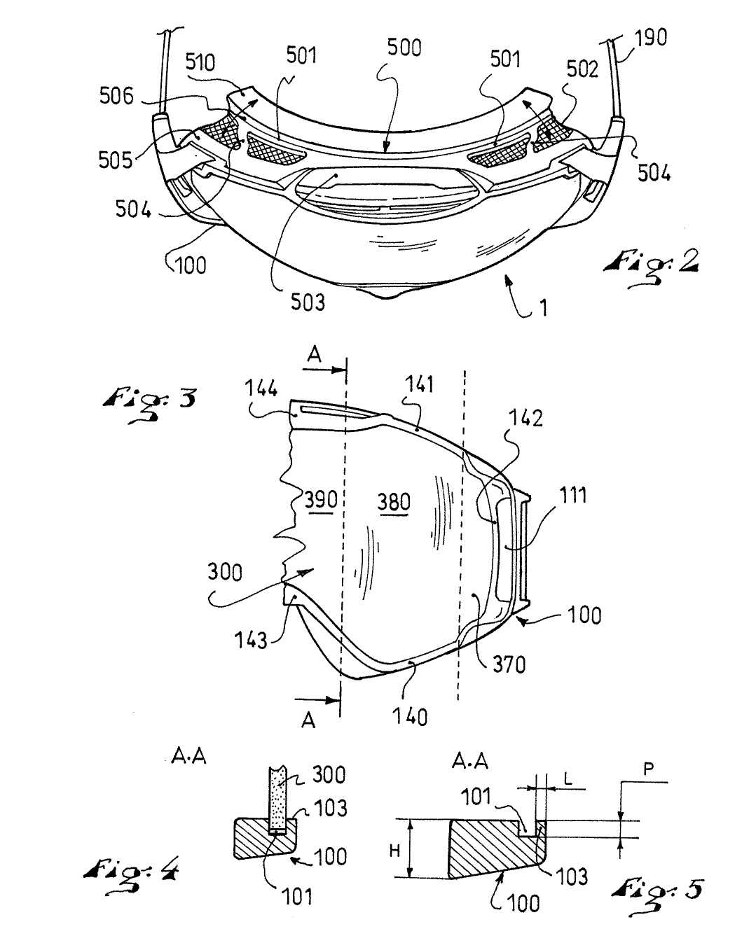

FIG. 3 is a front view of the left half of the frame 100 provided with the lens 300. The protective lens 300 closes, or blocks, the opening 102 in order to protect the user's eyes from external attacks, such as wind, snow, and/or water. The lens 300 is transparent and, in a known manner, can have light filtering properties in order to protect the user's eyes against solar radiation, for example. For example, the lens 300 can be structured to filter ultraviolet radiation.

The lens 300 has two vision zones 380 adapted to be positioned opposite the user's eyes when the mask is worn. The two vision zones 380 are connected to one another by a median portion 390, or median zone. The median portion 390 is structured and arranged to be positioned directly above the nose of the user. In a known manner, the lens 300 can form a continuous wall extending between the viewing zones 380 and passing through the median portion 390 in order to optimize the viewing angle of the user in the area of the median portion. The lens 300 also has peripheral vision zones 370 provided in its lateral ends, or lateral end regions.

As shown in FIG. 3, e.g., the frame 100 has an upper median portion 144, lateral upper portions 141, lateral end portions 142, lateral lower portions 140 and a median lower portion 143. The median lower portion 143 is notched, or upwardly concave, to allow passage of the user's nose.

FIGS. 4 and 5 are side cross-sectional views of the frame 100 in the area of a vision zone, in the presence and absence, respectively, of the lens 300. The frame 100 has a groove 101 housing the edge of the lens 300. The lens 300 can advantageously be fixed to the frame 100 by being snap-fastened, or snap-fitted, into the groove 101. The groove 101 can advantageously extend all around the lens 300, over the entire periphery of the opening 102, to ensure an optimal retention of the lens 300.

At the vertical extents of the aforementioned vision zones 380 of the lens 300, that is to say in the lower portion 140 and upper portion 141, the groove 101 has a depth P (see FIG. 5) of less than 2.5 mm, advantageously less than 2 mm and, in a particular embodiment, less than 1.5 mm. Thus, the viewing angle of the user at the vertical extents of the zones 380 can be substantially improved, because the height of the lens 300 is increased for a given space requirement of the mask 1. Advantageously, the groove 101 has such a depth P within the entire periphery of the opening 102, in order to optimize the viewing angle of the user for the entire field of vision.

The use of such depth of the groove 101 is made possible in particular due to the use of a frame 100 made of a rigid material. A groove 101 of lesser depth can thus be used to fix the lens 300 due to the lower frame 100 being subject to less deformation. Furthermore, a rigid frame 100 makes it possible to use a lens 300 made of a relatively rigid material, because the lens does not undergo significant deformation in view of the frame. Advantageously, the material selected to make the frame 100 has a modulus of elasticity greater than 1500 MPa, or, in a particular embodiment, greater than 2500 MPa or, in another embodiment, greater than 4000 MPa. Such a material can be a synthetic material, reinforced or not reinforced, such as with fiber (i.e., fiber-reinforced resin). For example, the frame 100 can be made of polyamide, polyurethane, or other plastic material. A polyamide such as that distributed under the trade name Grilamid® TR55 or Grilamid® TR90, for example, can be used. Furthermore, the frame 100 can have a hardness greater than 70 Shore D or, in another embodiment, greater than 80 Shore D.

Due to its rigidity, the frame 100 can have other constructional dimensions and configurations making it possible to increase the viewing angle of the user.

As illustrated in FIGS. 4 and 5, the frame 100 has an edge 103, or rim, defining a front stop of the groove 101. The rim 103 has a thickness L that is advantageously less than 2.5 mm or, in a particular embodiment, less than 2 mm or, in another embodiment, less than or equal to 1.5 mm, in the vertical extension, at the top and bottom, of the vision zone 380, in the portions 140 and 141. The rim 103 can have such a thickness dimension, i.e., a thickness in a generally front-to-back direction, over the entirety of the periphery of the opening 102.

To further maximize the viewing angle of the user, the cross section of the frame 100 in the portions 140 and 141, i.e., the bottom and top peripheral portions of the frame, has a height H less than 6 mm or, in a particular embodiment, less than 5 mm or, in another embodiment, less than or equal to 4 mm.

FIG. 16 shows the mask of FIG. 1 in cross section along the plane C. The plane C corresponds to a vertical plane intersecting the mask substantially at the center of one of the vision zones. The measurement of the free portion of the lens in the area of this plane makes it possible to determine the vertical extension of the field of vision. This measurement is represented by the arrow “c” in FIG. 16. Two other measurements are made in the area of the plane C, namely the height “a” of the frame 100 and the distance “b” separating the two inner edges 109 of the rim 103 of the frame. More specifically, the measurement “a” corresponds to the total height of the frame. Indeed, although this is not the case for the embodiment shown in FIG. 1, the total height of the mask, within the scope of the invention, i.e., according to at least one embodiment of the invention, can be slightly greater than the height of the frame.

In a mask, or goggles or protective eyewear, according to the invention, the vertical extension at the center of the vision zones is greater than 78% of the height of the frame measured in the same area. In a particular embodiment, this ratio is increased to 80%, for example by reducing the depth of the groove and/or by increasing the stiffness, i.e., the rigidity, of the frame.

For the mask shown in FIGS. 1 and 16, the vertical extension “c” of the field of vision is substantially greater than 86%.

Furthermore, from an aesthetic point of view, the shallowness of the groove receiving the lens has a very notable consequence: the lens of the mask represents the largest portion of the front surface. In fact, the distance “b” separating the inner edges of the frame is relatively long compared to the height “a” of the mask. In a mask of the invention, “b” is greater than 85% of the value “a” or, in a particular embodiment, greater than 88%. For example, the mask shown in FIGS. 1 and 16 has a b/a ratio that is slightly greater 93%. It should be noted that the b/a ratio would correspond to the ratio of the vertical extension of the field of vision to the height of the mask if the lens had only one wall.

The “wide field of vision” characteristic of the mask of the invention is also due to the fact that the height “d” from the frame in the area of the plane C is less than 5 mm or, in a particular embodiment, less than 3 mm, as in the embodiment described here.

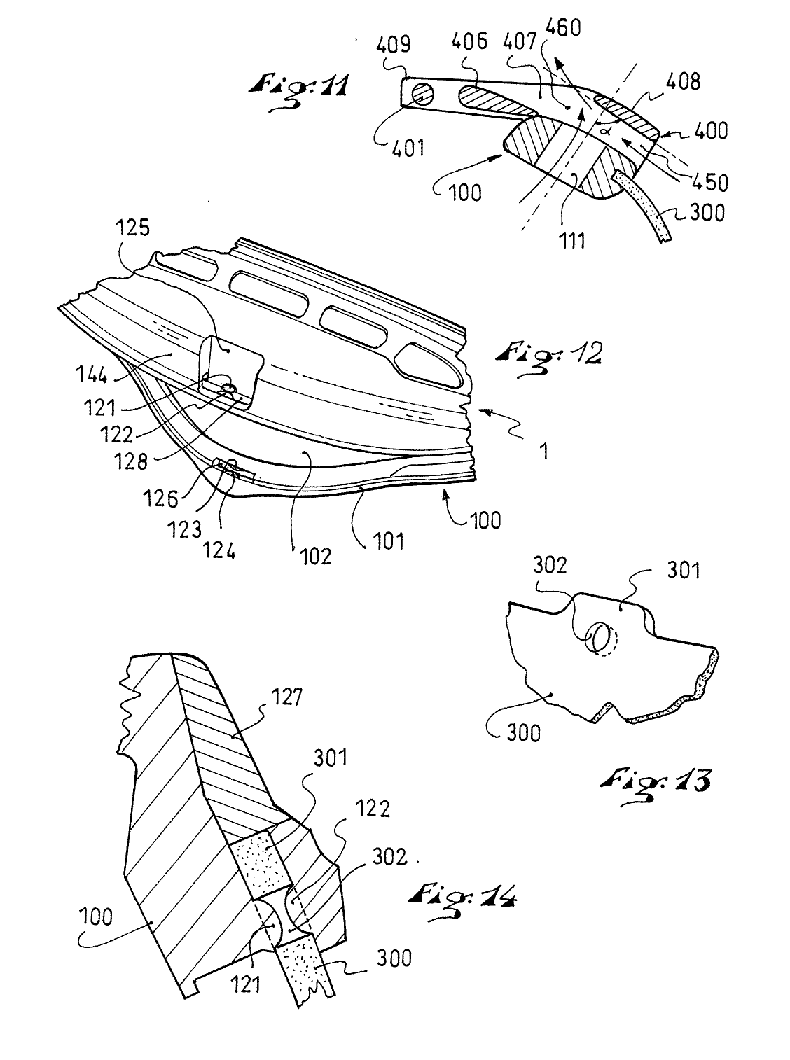

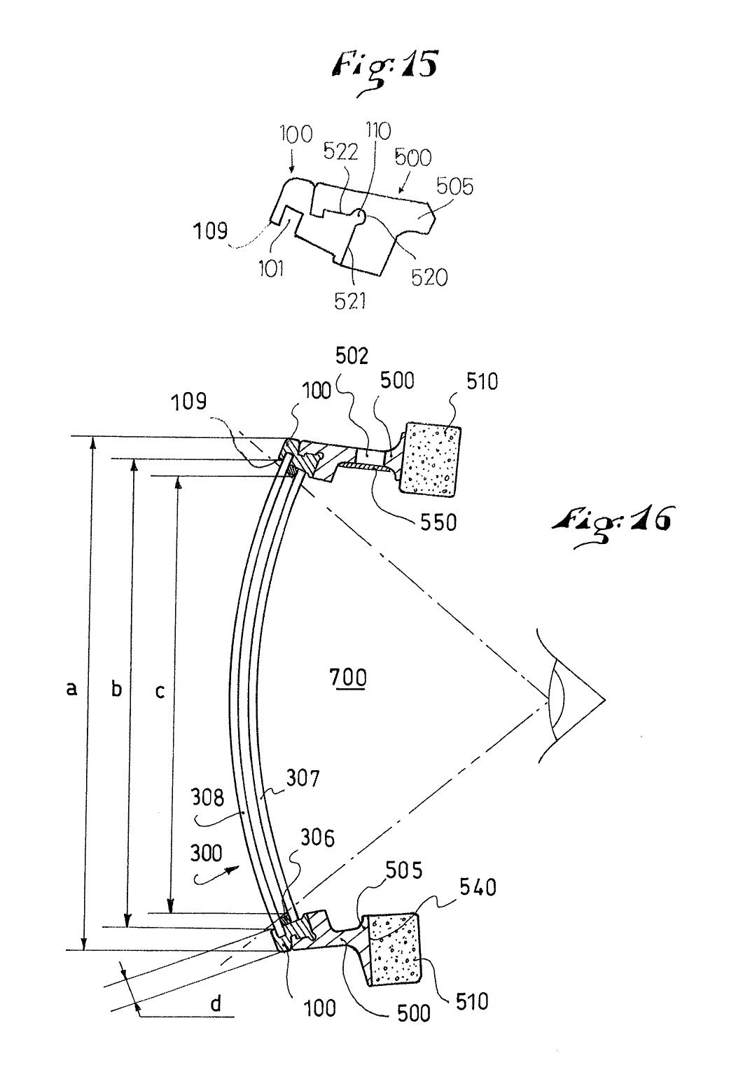

FIGS. 12 and 13 illustrate an alternative embodiment for improving a structure and method of fixing the lens 300 by snap-fastening it into the frame 100. In this example, the lens 300 has an opening in its upper median edge and lower median edge, respectively. FIG. 13 illustrates a portion of the lens 300, in the area of its upper opening 302. The edge of the lens 300 forms a projection 301 directly above the opening 302.

The frame 100 has an opening 128 in its upper portion 144. The orifice 128 is arranged in alignment with the groove 101. The opening 128 is advantageously arranged in the area of a recess 125 of the frame 100. The recess 125 can be shaped to receive a patch 127 comprising a logo. Bulges 121 and 122 are provided on both sides of the opening 128 and project within the opening. The frame 100 also has an opening 126 in its lower portion. Bulges 123 and 124 are provided on both sides of the opening 126 and project within the opening.

As illustrated in FIG. 14, the projection 301 is housed within the opening 128 when the edge of the lens 300 is being inserted into the groove 101. The nubs 121 and 122, or bumps or bulges, are then snapped into the opening 302 of the lens 300. The mechanical connection of the lens 300 to the frame 100 is thus improved. The patch 127, or insert part, is positioned in the recess 125 and hides the opening 128 and the projection 301.

According to another aspect of the invention shown in FIG. 1, the mask 1 has a deformable or flexible skirt 500, or shroud, attached to frame 100. The skirt 500 extends to the rear of the frame 100 and demarcates a second opening positioned in alignment with the opening 102. The skirt 500 is made of flexible material. The skirt 500 is formed from a material that is different from that of the frame and has a hardness well below, or less than, that of the frame 100. The skirt 500 makes it possible to define the inner volume of the mask behind the lens, between the latter and the user's face. The skirt 500 is adapted to be able to deform sufficiently to adapt to the morphology of the user's face or to absorb impacts that are applied to the frame 100. Such a skirt 500 is therefore particularly appropriate in combination with the rigid frame 100 described hereinabove, which has a reduced ability to adapt to the morphology of the face and to absorb impacts. The skirt can be made of a material similar to that used for making the frame of the ski masks of the prior art.

The skirt 500 has a ring 505 fixed to the entire contour of the frame 100. The skirt 500 has another ring 506, arranged at the rear of the ring 505. Advantageously, the rings 505 and 506 are made all in one piece. The ring 506 has a contour substantially identical to that of the ring 505. The ring 506 therefore has a contour substantially identical to that of the frame 100, which prevents the frame 100 from coming into contact with the user's face during an impact. The ring 505 has a median section 503 via which it is attached to the frame 100. In practice, the median section 503 is common to the ring 505 and to the ring 506. The ring 506, in practice, has two median sections, one in the area of the forehead, and the other in the area of the user's nose. One median section 503 is thus interposed between the user's forehead and the frame 100, the other median section 503 being interposed between the user's nose and the frame 100, the median sections 503 making it possible to ensure that the mask is held in position relative to the user's face. The ring 506 has lateral sections 501 extending laterally from the median sections 503.

As illustrated by the arrows in FIG. 2, the lateral sections 501 are axially movable relative to the frame 100 so as to change the inner volume 700. The mobility of the lateral sections 501, combined with the flexibility of the material of the skirt 500, enables the mask 1 to adapt to the morphology of the user's face. To this end, the ring 506 can have a radius of curvature about a vertical axis that is less than the usual radius of curvature of the face of the user, in order to ensure that the ring 506 conforms to the shape of the face of all potential users. The deformation of the skirt makes it possible to increase the radius of curvature of the ring 506. In practice, a clearance is provided laterally between the lateral sections 501 and the ring 505, in order to allow a certain amplitude for deformation and shock absorption. In contrast, the median section 503 is substantially immovable relative to the frame 100. Thus, when compressed axially, the rear portion of the median section is subject to a limited displacement, which is well shorter than the displacement to which the lateral sections are subject, for an axial compression of the same amplitude.

In the example, the median section 503 forms a continuous block of material between the frame 100 and the lateral sections 501. Thus, the median sections 503 make it possible to ensure that the frame 100 is properly retained, without altering the user's wearing comfort. The use of a flexible material for the skirt 500 prevents impacts from reverberating roughly on the nose or forehead of the user via the median sections 503.

Although the exemplary embodiment illustrated in FIG. 2 includes two axially superimposed rings, one can use a single ring 506 attached to the frame 100 by its median section 503, and whose lateral sections 501 are spaced apart from the frame 100.

To limit the rearward displacement of the lateral sections 501, upper connection bridges 504 advantageously connect the lateral sections 501 to the ring 505. Such upper connecting members 504, or bars, also improve the retention of the frame 100 in position relative to the user's face, as well as the resistance to wear of the mask 1. To facilitate the movements of the sections 501, 504, the upper connection bridges are advantageously inclined relative to the axis or a vertical median plane of the mask, which promotes their flexing.

FIG. 17 shows a side view of the skirt 500, making it possible to see the portions of the skirt that are hidden by the outer flap 408. The lateral section 501 can be seen to be connected to the ring 505 via two lateral connection bridges 530. These lateral connection bridges have a shape that makes it possible to bring the ring 505 and the lateral section 501 closer to one another. Indeed, the central portion 531 of the lateral connection bridge 530 can easily be deformed because it works in bending when the lateral section 501 comes closer to the ring 505.

The mask 1 advantageously has a gasket 510 to further improve its adaptation to the morphology of the user's face. The gasket 510 forms a belt attached to the rear of the ring 506, on its contour 540. The gasket 510 defines a third opening positioned in axial alignment with the opening 102, and it is adapted to come into contact with the user's face. The gasket 510 can be made of foam, such as a soft, compressible foam.

The frame 100, the lens 300, the skirt 500, and the gasket 510 thus demarcate an inner volume of the mask 1. For example, the user can have prescription eyeglasses in this volume. The inner volume of the mask 1 is also used to reduce the occurrence of condensation on the inner surface of the lens 300.

The clearance 502 between the lateral sections 501 and the ring 505 is advantageously blocked by a breathable fabric 550, in order to allow moisture to escape from the mask while preventing the penetration of foreign bodies and debris. By breathable fabric is meant a fabric that can allow air or water vapor to pass therethrough.

The junction between the lateral sections 501 and the ring 505 and/or the frame 100 can also be carried out by pleats housed or positioned in the clearances, or by beams or struts arranged in the clearances.

According to still additional alternative embodiments, the clearances between the lateral sections 501 and the ring 505 and/or the frame 100 can be blocked by a foam, allowing moisture to escape from within the mask.

Advantageously, the material of the skirt 500 has a hardness at least three times less than the hardness of the frame 100 or, in particular embodiments, at least five times less. The hardness of the material of the skirt 500 can, for example, be less than or equal to 80 Shore A or, in another embodiment, less than or equal to 70 Shore A. The hardness of the skirt 500 can be between 55 and 70 Shore A, for example. The skirt 500 can be made of any suitable material, such as polyurethane.

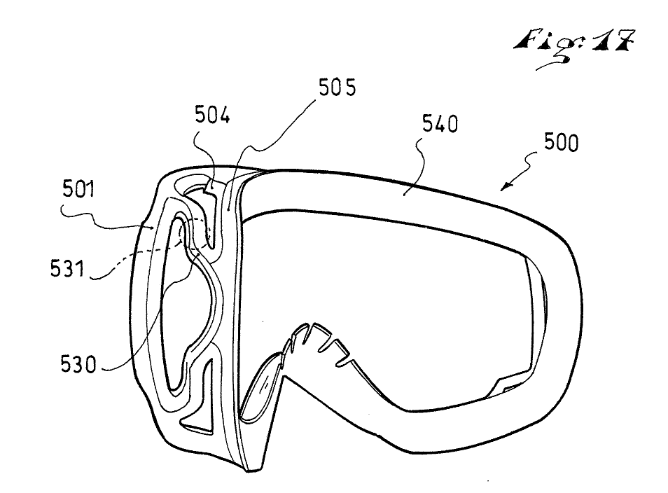

FIG. 15 shows a partial cross-sectional view of the assembly between the frame 100 and the skirt 500. The skirt is provided with of a groove 520, over a large portion of the outer surface of the ring 505, whereas the frame 100 is extended, on its surface opposite that which is provided with the groove 101, by means of a nub or bulge 110 having a shape complementary to that of the groove 520. The inner dimensions of the groove 520, and in particular the angle formed between the two flat surfaces 521, 522 which extend the portion with circular cross section of the groove 520, are slightly less than those of the bulge 110. Thus, given the difference in flexibility between the materials of the skirt 500 and of the frame 100, a clip-on connection, or snap-fit connection, is made between these two elements. This snap-fit connection can be completed by the application of glue and/or a plurality of screws through the interface between the skirt 500 and the frame 100, in the area of the flat faces 521, 522. In alternative embodiments, the snap-fit connection is replaced by at least any of gluing, welding, and screws.

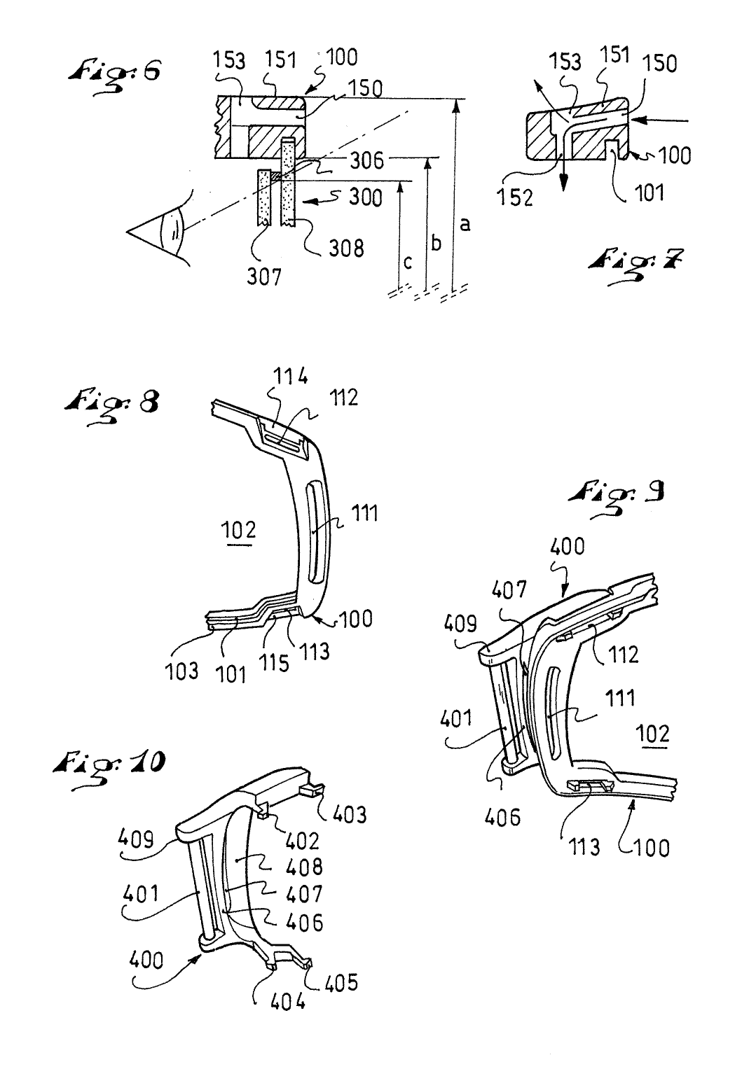

For the embodiment illustrated in FIG. 1, the portion of the lens 300 closing the opening 102 is devoid of any through-hole. Thus, the viewing angle of the user is not affected by such openings. The absence of such openings is particularly advantageous when the lens 300 has a plurality of superimposed walls, as shown in FIG. 6. In the example of FIG. 6, two walls 307 and 308 are axially superimposed. The wall 308 is fixed in the groove 101 and is adapted to dampen impacts and to filter a portion of the solar radiation. The wall 307 is fixed to the wall 308 via a peripheral joint 306 or connection. The peripheral joint 306 provides sealing between the walls 307 and 308. Ventilation holes extending through the lens should, in practice, extend through the wall 308 and should be located outside of the joint 306, such as beyond the periphery of the joint, in order to ensure sealing between the walls 307 and 308. In the absence of such openings, the joint 306 can be attached closer to the groove 101 of the frame 100, which increases the viewing angle of the user.

The frame 100 advantageously has a front opening 150 provided in its portion 144, arranged above the median portion 390 of the lens 300. Such front opening 150 arranged in the median portion of the frame 100 makes it possible not to hinder or obstruct the user's vision in the vision zones 380. The frame 100 has a front deflector 151 arranged on its front surface. The front deflector 151 demarcates the upper portion of the opening 150. Passageways 152 are arranged at the rear of the opening 150. The passageways 152 enable the inner volume of the mask to communicate with the outside. The outer inlets of the passageways 152 communicate with the opening 150. The passageways 152 open out into the inner volume of the mask, at the rear of the lens 300. An opening 153 is provided at the rear of the front deflector 151, directly above the passageways 152.

As illustrated in FIG. 7, during use, as the user moves forwardly, air rushes into the opening 150. The front deflector 151 channels the flow formed in the opening 150, such that a flow is formed inward of the mask 1 through the passageways 152. The opening 153 makes it possible to limit the pressure within the mask by allowing airflow out toward the top of the mask. The front portion of the front deflector 151 is advantageously raised relative to its rear portion, so as to guide the air coming in through the opening 150 toward the passageways 152. This structure for introducing air inside the mask 1 is advantageously combined with the openings 111, or orifices, and with the lateral deflector 400 (described in detail hereinafter) to generate an airflow going from the passageways 152 to the openings 111, which promotes the evacuation of water vapor from the mask 1.

According to another aspect of the invention, illustrated in more detail in FIGS. 8 to 11, the opening 111, or orifice, of the frame 100 enables the interior of the mask to communicate with the exterior of the mask. The opening 111 opens out inside the mask 1, at the rear of the lens 300. In this case, the opening 111 is provided in the lateral end portion 142 of the frame 100. Because the opening 111 is arranged in the area of one lateral end of the frame 100, it does not reduce the viewing angle of the user in the peripheral portion. To optimize suction without affecting the peripheral field of vision, the opening 111 extends over most of the height of the frame 100. The mask 1 includes a lateral deflector 400 fixed to the frame 100. The lateral deflector 400 is structured and arranged so as to be opposite the opening 111. The lateral deflector 400 is configured to channel airflow coming from the front of the mask when the user moves, in order to form an airflow tangentially to the outer inlet of the opening 111, such that the airflow formed creates a depression, or low pressure, by means of the venturi effect in the area of the outer inlet of the opening 111. The low pressure makes creates the formation of air flowing from the inside of the mask to the outside through the opening 111.

The lateral deflector 400, also referred to as the diffuser, has an outer flap 408 or blade arranged so as to be opposite the opening 111, slightly ahead of the latter. The outer flap 408 extends over the entire height of the orifice 111. The outer flap 408 has an inner surface guiding the airflow formed tangentially to the outer inlet of the orifice 111. The lateral deflector 400 has an inner flap 406 or blade positioned at the rear of the outer flap 408. The inner flap 406 extends rearward from the rear edge of the outer opening of the first passageway. In this case, the frame 100 is flush with the inner flap 406 to form a substantially continuous surface for guiding the downstream portion of the airflow formed. The flaps 406 and 408 are separated by a passage 407 traversed by the airflow formed. In general, the lateral deflector 400 defines a passage 407 between the outer flap 408, on the one hand, and the frame 100 and the inner flap 406, on the other hand. The passage 407 enables airflow between an inlet opening 450 and an outlet opening 460. The inlet opening 450 is open toward the front surface of the mask so that, during use (i.e., when the user moves forward), air is forced into the passage 407. The outlet opening 460 is open toward the rear of the mask, so that the flow can be very fluid, with a high flow rate between the inlet opening 450 and the outlet opening 460. The orifice 111 enables the interior of the mask to communicate with the passage 407. Indeed, the orifice 111 opens out into the passage 407. The passage 407 has a reduced cross section in the area of the orifice 111, and an enlarged cross section in the area of its downstream end, in the area of the outlet opening. The combination of the flaps 406 and 408 forms a constriction of the airflow formed, substantially in the area of the outer inlet of the orifice 111.

The orifice 111 of the embodiment illustrated is demarcated by a passageway formed in the frame 100. This passageway extends between the interior and the exterior of the mask 1 along a substantially normal direction with respect to the inner surface for guiding the outer flap 408. The angle between the direction of extension of the passageway and the inner surface for guiding the outer flap 408 is represented by the angle α in FIG. 11.

The lateral deflector 400 of the example is snap-fastened to the frame 100. To this end, the lateral deflector 400 has a first tab, or lug, in the area of its upper end, provided with hooks 402 and 403. The lateral deflector 400 also has a second tab or lug in the area of its lower end, provided with hooks 404 and 405. The hooks 402, 403, 404, 405 extend through respective openings 112 and 113 of the frame 100. The openings 112 and 113 are provided in the upper portion and lower portion, respectively, of the portion 142 of the frame 100. The hooks 402 to 405 engage a rear surface of the frame 100. The frame 100 advantageously has recesses 114 and 115 for receiving the hooked-tabs 402, 403 and 404, 405, respectively, for fixing the lateral deflector 400. The frame 100 advantageously has a larger cross section in the area of the openings 112 and 113.

The lateral deflector 400 advantageously includes a structure configured to affix the strap 190. In this regard, the lateral deflector 400 has a stirrup 409 positioned at the rear of the inner flap 406. The connecting of the strap 190 is advantageously in alignment with the inner flap 406. A shaft 401 extends vertically in the stirrup and forms a support for connecting the strap 190. In the embodiment shown in FIG. 1, a connector 191 is pivotally mounted on the shaft 401. The strap 190 is crimped into the connector 191. According to alternative embodiments, the strap can be affixed onto the shaft 401 according to known structures and arrangements, such as being threaded around the shaft and folded back upon itself and affixed to an adjustable buckle.

In the example shown, the lens 300 has a radius of curvature of less than 400 mm or, in an alternative embodiment, less than 300 mm. The passageway demarcating the opening 111 extends along a normal direction with respect to the zone 370 of the lens, that is to say the zone contiguous to the opening 111.

As illustrated in FIG. 11, the venturi effect generated by the airflow formed in the passage 407 generates a vacuum in the opening 111, such that air is sucked from within the mask outward of the mask. The evacuation of any fog or water vapor present inside the mask 1 is thus improved. To promote the extraction of air from the mask, the conduit demarcating the orifice 111 advantageously extends along a direction forming a return relative to the flow formed. This direction forming a return can be identified by an acute angle α between the surface for guiding the outer flap 408 and the direction of extension of the passageway demarcating the orifice.

The invention illustratively disclosed herein suitably may be practiced in the absence of any element which is not specifically disclosed herein.

1. An eye protection mask for the practice of outdoor sports, said mask comprising:
an eye protection lens comprising:
two vision zones, each of the two vision zones having a central portion structured and arranged to be positioned opposite a respective one of a user's eyes;
a median portion connecting said two vision zones;
a peripheral frame demarcating a frame opening closed by the eye protection lens;
the frame being made of a rigid material;
the frame having a groove housing an edge of the lens;
a deformable skirt fixed to the frame and extending rearwardly from the lens;
the deformable skirt demarcating a skirt opening, said skirt opening being in alignment with the frame opening, said skirt opening being structured and arranged to be in contact with the user's face;
a distance separating two inner edges of the frame, along a plane extending within an area of a central portion of at least one of the vision zones, is greater than 85% of the total height of the frame in said area.

2. An eye protection mask according to claim 1, wherein:
the vertical extension of the field of vision, in the area of the central portion of the vision zones, is greater than 78% of a height of the frame in said area.

3. An eye protection mask according to claim 1, wherein:
the groove has a depth of less than 2.5 mm in the vertical extension of the vision zones of the lens.

4. An eye protection mask according to claim 1, wherein:
the frame has a rim defining a front stop of said groove;
the rim has a thickness, in a generally front-to-back direction, of less than 2.5 in the vertical extension of the vision zones of the lens.

5. An eye protection mask according to claim 1, wherein:
in cross section, each of the top and bottom peripheral portions of the frame has a height of less than 6 mm in the vision zones of the lens.

6. An eye protection mask according to claim 1, wherein:
the lens is connected by means of a snap-fit into the groove of the frame.

7. An eye protection mask according to claim 1, wherein:
the groove extends over an entire periphery of the frame opening.

8. An eye protection mask according to claim 1, wherein:
the frame is made of a synthetic material having a modulus of elasticity greater than 1500 MPa.

9. An eye protection mask according to claim 1, wherein:
the frame has a hardness greater than 70 Shore D.

10. An eye protection mask according to claim 1, wherein:
within the frame opening, the lens is free of any through-hole;
the frame has a passageway opening out at a rear of the lens and arranged above the median portion of the lens.

11. An eye protection mask according to claim 8, further comprising:
a front deflector arranged on a front surface of the frame;
the front deflector being structured and arranged to direct an axial airflow toward an outer inlet of said passageway.

12. An eye protection mask according to claim 1, wherein:
the lens has two walls superimposed and affixed by a peripheral seal attached to said groove.
